(19) United States
(12) Patent Application Publication
(10) Pub. No.: US 2005/0048645 A1
(43) Pub. Date: Mar. 3, 2005

(54) Method of treating or preventing disease characterized by cryptococcus neoformans infection

(76) Inventor: Elaine K. Thomas, Seattle, WA (US)

Correspondence Address: IMMUNEX CORPORATION;LAW DEPARTMENT, 1201 AMGEN COURT WEST, SEATTLE, WA, 98119, US

(21) Appl. No.: 10/451,200

(22) PCT Filed: Dec. 14, 2001
(86) PCT No.: PCT/US01/48288

Related U.S. Application Data
(60) Provisional application No. 60/259,653, filed on Jan. 4, 2001.

(51) Int. Cl. A61K 45/00; C12N 5/06

(52) U.S. Cl. 435/325

(57) ABSTRACT

Antigen-expressing, activated dendritic cells are disclosed. Such dendritic cells are used to present Cryptococcus neoformans antigens to T cells, and can be useful in vaccination or treatment protocols. Other cytokines can be used in separate, sequential or simultaneous combination with the activated, antigen-pulsed dendritic cells. Also disclosed are methods for stimulating an immune response using the antigen-expressing, activated dendritic cells.

### CROSS REFERENCE TO RELATED APPLICATIONS

This application is a national-stage application under 35 U.S.C. § 371 of international application PCT/US01/48288, filed on Dec. 14, 2001, designating the United States and published in English on Aug. 29, 2002; which claims the benefit under 35 U.S.C. § 119 of U.S. Provisional Application Ser. No. 60/259,653, filed Jan. 4, 2001, the disclosure of which is incorporated herein by reference.

### FIELD OF THE INVENTION

The present invention relates to methods of enhancing a lymphocyte-mediated immune response against C. neoformans, and to dendritic cell populations useful in the manipulation of cellular and humoral immune responses against C. neoformans.

### BACKGROUND OF THE INVENTION

Vaccination is an efficient means of preventing death or disability from infectious diseases. Despite the successes achieved with the use of vaccines, however, there are still many challenges in the field of vaccine development. One such difficulty is lack of immunogenicity in an antigen, i.e., the antigen is unable to promote an effective immune response against the pathogen. Lack of suitable adjuvants (substances that enhance, augment or potentiate an immune response) also poses a challenge for development of effective vaccination protocols for numerous antigens.

Cryptococcus neoformans is an encapsulated yeast that causes opportunistic infections, often fatal, in immunocompromised individuals (for examples, AIDS patients). Primary infection is thought to occur by inhalation of C. neoformans; elimination of the infection (or at least prevention of disseminated infection) is believed to require an effective, specific cell-mediated immune response. Development of a specific, T_{H}1 cell-mediated immune response is governed by the cytokines secreted by the various cells of the immune systems that encounter C. neoformans.

Dendritic cells, a heterogeneous cell population with distinctive morphology and a widespread tissue distribution, are referred to as “professional” antigen presenting cells, and have a high capacity for sensitizing MHC-restricted T cells. Thus, there is growing interest in using dendritic cells ex vivo as tumor or infectious disease vaccine adjuvants (see, for example, Romani, et al., J. Exp. Med., 180:83, 1994). Therefore, an agent that enhanced the ability of dendritic cells to stimulate an immune response would be of wide importance.

### SUMMARY OF THE INVENTION

The present invention pertains to a method of activating dendritic cells to enhance antigen presenting capacity, and the use of such activated cells in preventing or treating disease caused by the fungus Cryptococcus neoformans.

The invention also provides a method of generating large quantities of antigen-presenting dendritic cells ex vivo. Following collection of an individual's CD34⁺ hematopoietic progenitors and stem cells, cytokines such as granulocyte-macrophage colony stimulating factor (GM-CSF) and flt-3 ligand (FL) can be used to expand the cells in vitro and to drive them to differentiate into cells of the dendritic cell lineage. Cytokines can also be used to increase the numbers of CD34⁺ cells in circulation prior to collection.

The resulting dendritic cells are exposed to C. neoformans antigen, and allowed to process the antigen (this procedure is sometimes referred to in the art as “antigen-pulsing”). The antigen-pulsed (or antigen-presenting) dendritic cells are then activated with a CD40 binding protein, and subsequently administered to the individual. For small, peptide antigens that do not require processing, the dendritic cells are contacted with a CD40 binding protein (which increases the numbers of MHC molecules on the dendritic cells) prior to exposing them to antigen.

An alternate method for preparing dendritic cells that present antigen is to transfect the dendritic cells with a gene encoding a C. neoformans antigen or a specific polypeptide derived therefrom. Once the dendritic cells express the antigen in the context of MHC, the dendritic cells are activated with a CD40 binding protein, and subsequently administered to the individual to provide a stronger and improved immune response to the antigen.

The activated antigen-presenting dendritic cells can also be used as a vaccine or vaccine adjuvant and can be administered prior to, concurrently with or subsequent to administration of C. neoformans antigen. Moreover, the dendritic cells can be administered to the individual prior to, concurrently with or subsequent to administration of cytokines that modulate an immune response, for example a CD40 binding protein (i.e., soluble CD40L). Additional useful cytokines include, but are not limited to, Interleukins (IL) 1, 2, 4, 5, 6, 7, 10, 12 and 15, colony stimulating factors (CSF) such as GM-CSF, granulocyte colony stimulating factor (G-CSF), or GM-CSF/IL-3 fusion proteins, or other cytokines such as TNF-alpha or c-kit ligand. Moreover, biologically active derivatives of these cytokines; and combinations thereof will also be useful.

The invention also provides for the ex vivo preparation of C. neoformans-specific T cells. Following the procedures described above for preparing large numbers of antigen-presenting dendritic cells ex vivo, the collected antigen-presenting dendritic cells are used to generate antigen-specific T cells from naive T cells that have been collected from the individual. After the antigen has been adequately presented to the T cells generated, the antigen-specific T cells can be administered to the individual.

Culture conditions can be selected to preferentially yield the type of T cell most desired (T_{H}1 versus T_{H}2). The C. neoformans-specific T cell response can be augmented by administering, simultaneously, separately or sequentially, agents that act on T cells (for example, IL-12, IL-15, Interferon-gamma, OX40 ligand, 4-1BB ligand, CD40L, or agonistic antibodies that mimic any of the foregoing).

### BRIEF DESCRIPTION OF THE DRAWINGS

FIG. 1 presents the nucleotide and amino acid sequence of human granulocyte-macrophage colony stimulating factor.

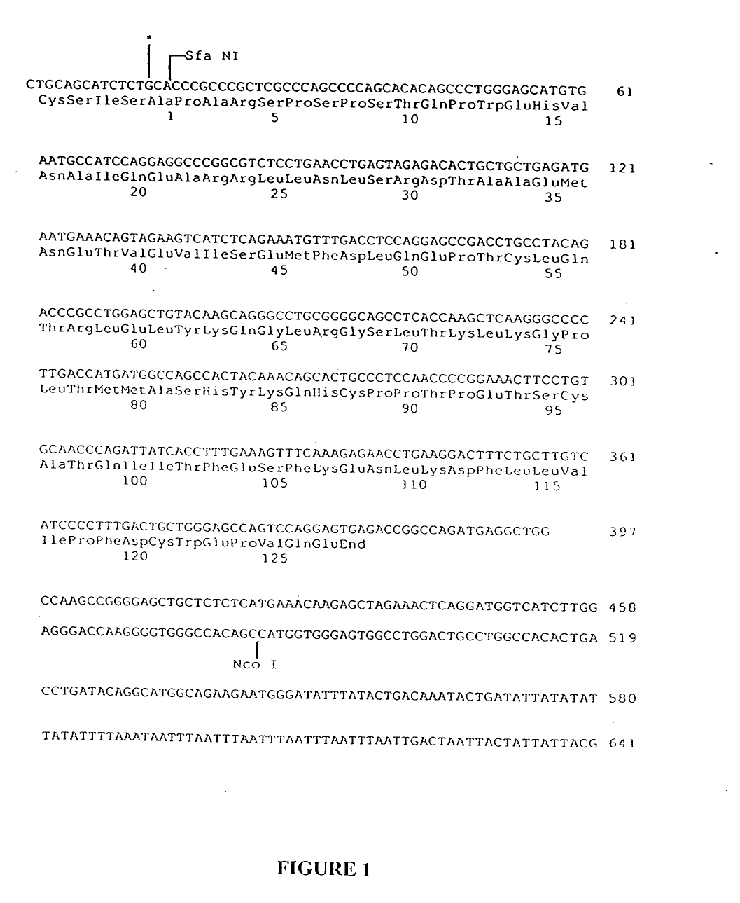

### DETAILED DESCRIPTION OF THE INVENTION

The invention is directed to the use of CD40L to activate C. neoformans-pulsed dendritic cells. Activation enhances the ability of the dendritic cells to present antigen to lymphoid cells, and thus augments the immune response against the antigen. Another embodiment of the invention is the isolation and use of activated, C. neoformans-pulsed dendritic cells as vaccines or vaccine adjuvants. The activated, antigen-pulsed dendritic cells may also be used ex vivo to generate antigen-specific T cells.

Dendritic Cells

Dendritic cells comprise a heterogeneous cell population with distinctive morphology and a widespread tissue distribution. The dendritic cell system and its role in immunity is reviewed by Steinman, R. M., Annu. Rev. Immunol., 9:271-296 (1991), incorporated herein by reference. The cell surface of dendritic cells is unusual, with characteristic veil-like projections, and is characterized by having the cell surface markers CD1a⁺, CD4⁺, CD86⁺, or HLA-DR⁺. Dendritic cells have a high capacity for sensitizing MHC-restricted T cells and are very effective at presenting antigens to T cells in situ, both self-antigens during T cell development and tolerance and foreign antigens during immunity.

Because of their effectiveness at antigen presentation, there is growing interest in using dendritic cells ex vivo as tumor or infectious disease vaccine adjuvants (see, for example, Romani, et al., J. Exp. Med., 180:83 (1994). The use of dendritic cells as immunostimulatory agents has been limited due to the low frequency of dendritic cells in peripheral blood, the limited accessibility of lymphoid organs and the dendritic cells' terminal state of differentiation. Dendritic cells originate from CD34+ bone marrow or peripheral blood progenitors and peripheral blood mononuclear cells, and the proliferation and maturation of dendritic cells can be enhanced by the cytokines GM-CSF (sargramostim, Leukine®, Immunex Corporation, Seattle, Wash.), TNF-alpha, c-kit ligand (also known as stem cell factor (SCF), steel factor (SF), or mast cell growth factor (MGF)) and interleukin4. Recently, FL (U.S. Pat. No. 5,554,512, issued Sep. 10, 1996) has been found to stimulate the generation of large numbers of functionally mature dendritic cells, both in vivo and in vitro (U.S. Ser. No. 09/448,378, filed Nov. 23, 1999).

Ex vivo Culture of Dendritic Cells

A procedure for ex vivo expansion of hematopoietic stem and progenitor cells is described in U.S. Pat. No. 5,199,942, incorporated herein by reference. Other suitable methods are known in the art (i.e., the well-known technique of ex vivo culture of peripheral blood mononuclear cells in IL-4 and GM-CSF [and/or other cytokines] to yield antigen-presenting cells). Briefly, ex vivo culture and expansion comprises: (1) collecting CD34⁺ hematopoietic stem and progenitor cells from a patient from peripheral blood harvest or bone marrow explants; and (2) expanding such cells ex vivo. In addition to the cellular growth factors described in U.S. Pat. No. 5,199,942, other factors such as FL, IL-1, IL-3 and c-kit ligand, can be used.

Stem or progenitor cells having the CD34 marker constitute only about 1% to 3% of the mononuclear cells in the bone marrow. The amount of CD34⁺ stem or progenitor cells in the peripheral blood is approximately 10- to 100-fold less than in bone marrow. Cytokines such as FL may be used to increase or mobilize the numbers of dendritic cells in vivo. Increasing the quantity of an individual's dendritic cells may facilitate antigen presentation to T cells for antigen(s) that already exists within the patient, such as a tumor antigen, or a bacterial or viral antigen. Alternatively, cytokines may be administered prior to, concurrently with or subsequent to administration of an antigen to an individual for immunization purposes.

Peripheral blood cells are collected using apheresis procedures known in the art. See, for example, Bishop et al., Blood, vol. 83, No. 2, pp. 610-616 (1994). Briefly, peripheral blood progenitor cells (PBPC) and peripheral blood stem cells (PBSC) are collected using conventional devices, for example, a Haemonetics Model V50 apheresis device (Haemonetics, Braintree, Mass.). Four-hour collections are performed typically no more than five times weekly until approximately 6.5×10⁸ mononuclear cells (MNC)/kg are collected. The cells are suspended in standard media and then centrifuged to remove red blood cells and neutrophils. Cells located at the interface between the two phases (the buffy coat) are withdrawn and resuspended in HBSS. The suspended cells are predominantly mononuclear and a substantial portion of the cell mixture are early stem cells.

A variety of cell selection techniques are known for identifying and separating CD34⁺ hematopoietic stem or progenitor cells from a population of cells. For example, monoclonal antibodies (or other specific cell binding proteins) can be used to bind to a marker protein or surface antigen protein found on stem or progenitor cells. Several such markers or cell surface antigens for hematopoietic stem cells (i.e., flt-3, CD34, My-10, and Thy1) are known in the art, as are specific binding proteins therefore (see for example, U.S. Ser. No. 08/539,142, filed Oct. 4, 1995).

In one method, antibodies or binding proteins are fixed to a surface, for example, glass beads or flask, magnetic beads, or a suitable chromatography resin, and contacted with the population of cells. The stem cells are then bound to the bead matrix. Alternatively, the binding proteins can be incubated with the cell mixture and the resulting combination contacted with a surface having an affinity for the antibody-cell complex. Undesired cells and cell matter are removed providing a relatively pure population of stem cells. The specific cell binding proteins can also be labeled with a fluorescent label, e.g., chromophore or fluorophore, and the labeled cells separated by sorting. Preferably, isolation is accomplished by an immunoaffinity column.

Immunoaffinity columns can take any form, but usually comprise a packed bed reactor. The packed bed in these bioreactors is preferably made of a porous material having a substantially uniform coating of a substrate. The porous material, which provides a high surface area-to-volume ratio, allows for the cell mixture to flow over a large contact area while not impeding the flow of cells out of the bed. The substrate should, either by its own properties, or by the addition of a chemical moiety, display high-affinity for a moiety found on the cell-binding protein. Typical substrates include avidin and streptavidin, while other conventional substrates can be used.

In one useful method, monoclonal antibodies that recognize a cell surface antigen on the cells to be separated are typically further modified to present a biotin moiety. The affinity of biotin for avidin thereby removably secures the monoclonal antibody to the surface of a packed bed (see Berenson, et al., J. Immunol. Meth., 91:11, 1986). The packed bed is washed to remove unbound material, and target cells are released using conventional methods. Immunoaffinity columns of the type described above that utilize biotinylated anti-CD34 monoclonal antibodies secured to an avidin-coated packed bed are described for example, in WO 93/08268.

An alternative means of selecting the quiescent stem cells is to induce cell death in the dividing, more lineage-committed, cell types using an antimetabolite such as 5-fluorouracil (5-FU) or an alkylating agent such as 4-hydroxycyclophosphamide (4-HC). The non-quiescent cells are stimulated to proliferate and differentiate by the addition of growth factors that have little or no effect on the stem cells, causing the non-stem cells to proliferate and differentiate and making them more vulnerable to the cytotoxic effects of 5-FU or 4-HC. See Berardi et al., Science, 267:104 (1995), which is incorporated herein by reference.

Isolated stem cells can be frozen in a controlled rate freezer (e.g., Cryo-Med, Mt. Clemens, Mich.), then stored in the vapor phase of liquid nitrogen using dimethylsulfoxide as a cryoprotectant. A variety of growth and culture media can be used for the growth and culture of dendritic cells (fresh or frozen), including serum-depleted or serum-based media. Useful growth media include RPMI, TC 199, Iscoves modified Dulbecco's medium (Iscove, et al., F. J. Exp. Med., 147:923 (1978)), DMEM, Fischer's, alpha medium, NCTC, F-10, Leibovitz's L-15, MEM and McCoy's.

Particular nutrients present in the media include serum albumin, transferrin, lipids, cholesterol, a reducing agent such as 2-mercaptoethanol or monothioglycerol, pyruvate, butyrate, and a glucocorticoid such as hydrocortisone 2-hemisuccinate. More particularly, the standard media includes an energy source, vitamins or other cell-supporting organic compounds, a buffer such as HEPES, or Tris, that acts to stabilize the pH of the media, and various inorganic salts. A variety of serum-free cellular growth media is described in WO 95/00632, which is incorporated herein by reference.

The collected CD34⁺ cells are cultured with suitable cytokines, for example, as described herein, and in U.S. Ser. No. 09/448,378. CD34⁺ cells then are allowed to differentiate and commit to cells of the dendritic lineage. These cells are then further purified by flow cytometry or similar means, using markers characteristic of dendritic cells, such as CD1a, HLA DR, CD80 and/or CD86. The cultured dendritic cells are exposed to an antigen, for example, a tumor antigen or an antigen derived from a pathogenic or opportunistic organism, allowed to process the antigen, and then cultured with an amount of a CD40 binding protein to activate the dendritic cell. Alternatively, the dendritic cells are transfected with a gene encoding an antigen, and then cultured with an amount of a CD40 binding protein to activate the antigen-presenting dendritic cells.

The activated, antigen-carrying dendritic cells are then administered to an individual in order to stimulate an antigen-specific immune response. Alternatively, the dendritic cells can be administered to the individual along with additional antigen (prior to, concurrently with, or subsequent to, antigen administration). Alternatively, T cells may be collected from the individual and exposed to the activated, antigen-carrying dendritic cells in vitro to stimulate antigen-specific T cells, which are administered to the individual.

Appropriate culture conditions can be selected to enhance the growth of preferred subtypes of T cells. Type 1 T_{H} cells (T_{H}1 cells) mediate delayed type hypersensitivity (DTH), and secrete Interferon-γ (IFN-γ) and Interleukin-2 (IL-2), while Type 2 T_{H} cells (T_{H}2 cells) secrete primarily Interleukins 4, 5 and 10 (IL-4, IL-5 and IL-10, respectively) and provide B cell help. Interleukin-12 (IL-12) is crucial in influencing the development of a T_{H}1 response, whereas IL-4 appears to result in the development of a T_{H}2 response.

Useful Cytokines

Various cytokines will be useful in the ex vivo culture of dendritic cells. FL refers to a genus of polypeptides that are described in EP 0627487 A2 and in U.S. Pat. No. 5,554,512, issued Sep. 10, 1996, both incorporated herein by reference. A human FL cDNA was deposited with the American Type Culture Collection, Rockville, Md., USA (ATCC) on Aug. 6, 1993 and assigned accession number ATCC 69382. IL-3 refers to a genus of interleukin-3 polypeptides as described in U.S. Pat. No. 5,108,910, incorporated herein by reference. A DNA sequence encoding human IL-3 protein suitable for use in the invention is publicly available from the American Type Culture Collection (ATCC) under accession number ATCC 67747. c-kit ligand is also referred to as Mast Cell Growth Factor (MGF), Steel Factor or Stem Cell Factor (SCF), and is described in EP 423,980, which is incorporated herein by reference.

Other useful cytokines include Interleukin-4 (IL-4; Mosley et al., Cell 59:335 (1989), Idzerda et al., J. Exp. Med. 171:861 (1990) and Galizzi et al., Intl. Immunol. 2:669 (1990), each of which is incorporated herein by reference) and granulocyte-macrophage colony stimulating factor (GM-CSF; described in U.S. Pat. Nos. 5,108,910, and 5,229,496 each of which is incorporated herein by reference). Commercially available GM-CSF (sargramostim, Leukine®) is obtainable from Immunex Corp., Seattle, Wash.). Moreover, GM-CSF/IL-3 fusion proteins (i.e., a C-terminal to N-terminal fusion of GM-CSF and IL-3) will also be useful in ex vivo culture of dendritic cells. Such fusion proteins are known and are described in U.S. Pat. Nos. 5,199,942, 5,108,910 and 5,073,627, each of which is incorporated herein by reference. A preferred fusion protein is PIXY321 as described in U.S. Pat. No. 5,199,942.

In addition to their use in ex vivo culture of dendritic cells, cytokines will also be useful in the present invention by separate, sequential or simultaneous administration of a cytokine or cytokines with activated, antigen-pulsed dendritic cells. Preferred cytokines are those that modulate an immune response, particularly cytokines selected from the group consisting of Interleukins 1, 2, 3, 4, 5, 6, 7, 10, 12 and 15; granulocyte-macrophage colony stimulating factor, granulocyte colony stimulating factor; a fusion protein comprising Interleukin-3 and granulocyte-macrophage colony stimulating factor; Interferon-gamma; TNF; TGF-β; FL; soluble CD40 ligand; biologically active derivatives of these cytokines; and combinations thereof. Soluble CD83, described in U.S. Ser. No. 08/601,954, filed Feb. 15, 1996), and soluble CD40L (described in U.S. Ser. No. 08/477,733 and U.S. Ser. No. 08/484,624, both filed Jun. 7, 1995) are particularly preferred cytokines.

Useful cytokines act by binding a receptor present on the surface of a cell and transducing a signal. Moreover, additional binding proteins can be prepared, by methods that are known in the art, that bind appropriate cytokine receptors and transduce a signal to a dendritic cell. For example, WO 95/27062 describes agonistic antibodies to Flt-3, the receptor for FL, from which various Flt-3 binding proteins can be prepared. Additional useful cytokines include biologically active analogs of cytokines that are useful for culturing dendritic cells. Useful cytokine analogs have an amino acid sequence that is substantially similar to the native cytokine, and are biologically active capable of binding to their specific receptor and transducing a biological signal. Such analogs can be prepared and tested by methods that are known in the art and as described herein.

CD40/CD40 Binding Proteins

CD40 is a member of the tumor necrosis factor (TNF)/nerve growth factor (NGF) receptor family that has been found to be expressed on B lymphocytes, monocytes some epithelial cells and dendritic cells (Clark, Tissue Antigens, 36:33; 1990). This cell surface antigen has been shown to play an important role in B cell proliferation and differentiation, and in the growth of malignant cells upon which it is expressed. Moreover, CD40 is expressed on dendritic cells, and transduction of signal to dendritic cells through CD40 can enhance the ability of the dendritic cells to present antigen (U.S. Pat. No. 6,017,527, issued Jan. 25, 2000).

The ligand for CD40 (hereinafter “CD40L”) has been identified and characterized, and DNA encoding the same has been cloned from peripheral blood T cells (Spriggs et al, J. Exp. Med., 176:1453 (1992); Armitage et al, Nature, 357:80 (1992); and Armitage et al, U.S. Pat. Nos. 5,961,974, 5,962,406 and 5,981,724; each of which is incorporated by reference herein in its entirety). CD40L biological activity is mediated by binding of this cytokine with CD40, and includes B cell proliferation in the absence of any co-stimulus, and induction of antibody secretion from B cells, excluding IgE secretion, in the presence of cytokines.

Soluble CD40L and agonistic antibodies to CD40 (i.e., those that mimic the biological effects of CD40L) have been used in the treatment of diseases characterized by neoplastic cells that express CD40, such as B lymphomas, some melanomas and some carcinomas (U.S. Pat. No. 5,674,492). Similarly, soluble CD40L has been used to promote the proliferation and/or differentiation of CD40-bearing cells that are not B cells, e.g., sarcoma cells, as a means of directly treating the malignancy or as an adjunct to chemotherapy, or to increase the immune response of an immunosuppressed individual, such as a subject suffering from malignancy (U.S. Pat. No. 5,945,513).

As used herein, “CD40 binding protein” refers to a protein (or proteins) that specifically binds CD40 in a noncovalent interaction based upon the proper conformation of the CD40 binding protein and CD40 itself. Preferably, the CD40 binding protein has agonistic activity, that is, it mimics the native ligand for CD40 (CD40L) that is present on activated T cells by binding to, and transducing a signal to, a cell expressing CD40. Assays for any of the aforementioned biological activities of CD40L will be useful for assessing agonistic activity. Another way to measure agonistic activity is to assess a CD40 binding protein for the ability to inhibit binding of CD40 to CD40L. CD40 binding proteins that bind CD40 and inhibit binding of CD40 to CD40L, as determined by observing at least about 90% inhibition of the binding of soluble CD40 to CD40L, will have agonistic activity.

A variety of CD40 binding proteins may be employed in the present invention, including, for example, an antibody that binds CD40; full-length-membrane bound CD40L; a soluble extracellular region of a CD40L; a fusion protein comprising a CD40 binding region (or domain) from a CD40L or an antibody to CD40, fused to a second protein, for example, an immunoglobulin Fc domain or a zipper domain.

Antibodies to CD40 which can be employed in the present invention may be polyclonal or monoclonal. The particular agonistic CD40 antibody employed in the present invention is not critical thereto. Examples of such CD40 antibodies include HuCD40-M2 (ATCC No. HB11459) and HuCD40-M3, and antigen binding domains thereof. Additional CD40 mAbs which can be employed in the present invention may be generated using conventional techniques (see U.S. Pat. Nos. RE 32,011, 4,902,614, 4,543,439, and 4,411,993, which are incorporated by reference herein in their entirety. Useful agonistic antibodies may also be constructed utilizing recombinant DNA techniques to “humanize” a murine antibody, or prepare single-chain antibodies, as described in U.S. Pat. No. 5,801,227.

As used herein, “CD40L” refers to a genus of polypeptides which are capable of binding CD40, or mammalian homologs of CD40 and transducing a signal thereby. CD40L includes full-length CD40L polypeptide, as well as soluble CD40L polypeptides lacking transmembrane and intracellular regions, mammalian homologs of human CD40L, analogs of human or murine CD40L or derivatives of human or murine CD40L. Also included within the term CD40L are CD40L from other species, and analogs or derivatives thereof.

cDNAs encoding these ligands have been cloned and sequenced as described in the above cited Armitage et al. U.S. Pat. Nos. 5,961,974, 5,962,406 and 5,981,724 (hereinafter, the Armitage patents). As disclosed therein, full-length CD40L is a membrane-bound polypeptide with an extracellular region at its C terminus, a transmembrane region, and an intracellular region at its N-terminus. The nucleotide sequence and deduced amino acid sequence of representative murine and human CD40L cDNA is disclosed in the Armitage patents, and is hereby incorporated by reference. Human CD40L a peptide comprises amino acids 1 through 261 of the sequence of human CD40L disclosed therein.

A soluble version of CD40L can be made from the extracellular region, or a fragment thereof, using standard molecular biology techniques as disclosed in the Armitage patents, supra. Soluble CD40L comprises amino acids 35 through 261, 34 through 225, 113 through 261, 113 through 225, 120 through 261, or 120 through 225 of human CD40L. A preferred CD40L is soluble, oligomeric CD40L referred to as trimeric CD40L in the Armitage patents. Trimeric CD40L comprises a fragment of the extracellular domain of CD40L fused to a zipper domain (disclosed in the Armitage patents and hereby incorporated by reference) that facilitates trimerization. Preferably, the cysteine amino acid 194 of human CD40L is substituted with tryptophan.

CD40L comprising various additions or substitutions of amino acid residues or sequences, or deletions of terminal or internal residues or sequences not needed for biological activity or binding can be prepared as disclosed by Armitage et al. supra. Moreover, other analyses may be performed to assist the skilled artisan in selecting sites for mutagenesis. Such CD40L polypeptides include fragments of CD40L; peptides encoded by DNA which hybridizes to a DNA that encodes human CD40L, under stringent conditions (hybridization in 6×SSC at 63° C. overnight; washing in 3×SSC at 55° C.). Moreover, PCT/US92/03743 (the disclosure of which is hereby incorporated by reference herein in its entirety) discusses methods of selecting ligand agonists and antagonists. These and other forms of biologically active CD40L will be useful in the present invention.

Methods for expression of recombinant CD40L polypeptides are also described in the Armitage patents. Similar methods may be used for expression of other CD40 binding proteins. Moreover, numerous expression systems are known to those of routine skill in the art of molecular biology, including prokaryotic and eukaryotic expression systems. The expression system selected may affect the nature of the recombinant CD40 binding protein expressed. For example, CD40L expressed in mammalian expression systems (e.g., COS7 cells) may be similar to a native CD40L in molecular weight and glycosylation pattern, whereas CD40L expressed in yeast may be more highly glycosylated than native CD40L. Expression of CD40L in bacterial expression systems, such as E. coli, provides non-glycosylated molecules.

Once suitable CD40 binding proteins have been obtained, they may be isolated or purified by many techniques well known to those of ordinary skill in the art. Suitable techniques include peptide or protein affinity columns, HPLC or RP-HPLC, purification on protein A or protein G columns, or any combination of these techniques. Recombinant CD40 binding proteins can be prepared according to standard methods, and tested for binding specificity to the CD40 utilizing assays known in the art, including for example ELISA, ABC, or dot blot assays, as well by bioactivity assays such as those described for CD40 mAb.

Preparation of C. neoformans Antigen-expressing Dendritic Cells

Immunization is a centuries old, and highly effective, means of inducing a protective immune response against pathogens in order to prevent or ameliorate disease. The vaccines that have been used for such induction are generally live, attenuated microorganisms, or preparations of killed organisms or fractions thereof. Live, attenuated vaccines are generally thought to more closely mimic the immune response that occurs with a natural infection than do those prepared from killed microbes or non-infective preparations derived from pathogens (i.e., toxoids, recombinant protein vaccines). However, attenuated vaccines also present a risk of reversion to pathogenicity, and can cause illness, especially in immunocompromised individuals.

The encapsulated yeast C. neoformans provides an attractive target for development of immunization techniques that can be used to prevent infection in naive individuals, or stimulate an immune response that will reduce or eliminate cryptococcal disease in infected individuals. Several types of cryptococcal antigen are known to those of skill in the art (see, for example, Dixon et al., Med. Mycol. 36 Suppl. 1:57; 1998). One such antigen is a cryptococcal cell wall/membrane (CCWM) preparation (Mody et al., J. Infectious Disease 178:803; 1998). Additionally, cryptococcal mannoproteins include secreted proteins that can be purified from culture supernatants (Pitzurra et al., Infection and Immunity 68:558; 2000). Moreover, DNAs (or mRNAs) encoding desired cryptococcal antigens can be used as described below by transfecting antigen-presenting cells, or be used to obtain purified cryptococcal antigens by recombinant means.

Purified dendritic cells are pulsed with (exposed to) a desired cryptococcal antigen, to allow them to take up the antigen (or bind smaller peptides) in a manner suitable for presentation to other cells of the immune systems. Antigens are classically processed and presented through two pathways. Peptides derived from proteins in the cytosolic compartment are presented in the context of Class I MHC molecules, whereas peptides derived from proteins that are found in the endocytic pathway are presented in the context of Class II MHC. However, those of skill in the art recognize that there are exceptions; for example, the response of CD8⁺ tumor specific T cells, which recognize exogenous tumor antigens expressed on MHC Class I. A review of MHC-dependent antigen processing and peptide presentation is found in Germain, R. N., Cell 76:287 (1994).

Numerous methods of pulsing dendritic cells with antigen are known; those of skill in the art regard development of suitable methods for a selected antigen as routine experimentation. In general, the antigen is added to cultured dendritic cells under conditions promoting viability of the cells, and the cells are then allowed sufficient time to take up and process the antigen, and express antigen peptides on the cell surface in association with either Class I or Class II MHC, a period of about 24 hours (from about 18 to about 30 hours, preferably 24 hours). Dendritic cells may also be exposed to antigen by transfecting them with DNA encoding the antigen. The DNA is expressed, and the antigen is presumably processed via the cytosolic/Class I pathway.

After antigen has been processed, the dendritic cells are contacted with a DC maturation factor such as CD40L. CD40L and other DC maturation factors increase the numbers of MHC molecules (and costimulatory molecules such as CD80 and CD83) on the surface of the DC, thereby enhancing their antigen-presenting ability. Moreover, DC that have been exposed to maturation factors secrete cytokines that are indicative of activation (for example, IL-12, IL-15). CD4+ cells that are presented antigen by mature, activated DC will express IL-2, IL-4, and IFN-gamma, which act as growth factors for T cells. Accordingly, mature, activated dendritic cells are able to stimulate an effective, antigen-specific immune response.

Smaller antigens such as peptides do not require processing by the dendritic cell, but are bound to the appropriate MHC molecules upon exposure of the DC to the peptides. When a peptide antigen is used, it is advantageous to stimulate the maturation of the DC prior to exposure to the peptide antigen, in order to increase the numbers of available MHC molecules, and thereby enhance antigen-carrying capacity. The same DC maturation factors that are useful in stimulating the maturation of DC that have processed larger protein antigens will also be useful in augmenting the capacity of DC to present smaller peptide antigens.

Administration of Activated, Antigen-pulsed Dendritic Cells

The present invention provides methods of using therapeutic compositions comprising activated, antigen-pulsed dendritic cells. The use of such cells in conjunction with soluble cytokine receptors or cytokines, or other immunoregulatory molecules is also contemplated. The inventive compositions are administered to stimulate an immune response, and can be given by bolus injection, continuous infusion, sustained release from implants, or other suitable technique. Typically, the cells of the inventive methods will be administered in the form of a composition comprising the antigen-pulsed, activated dendritic cells in conjunction with physiologically acceptable carriers, excipients or diluents. Such carriers will be nontoxic to recipients at the dosages and concentrations employed. Neutral buffered saline or saline mixed with conspecific serum albumin are exemplary appropriate diluents.

For use in stimulating a certain type of immune response, administration of other cytokines along with activated, antigen-pulsed dendritic cells is also contemplated. Several useful cytokines (or peptide regulatory factors) are discussed in Schrader, J. W. (Mol Immunol 28:295; 1991). Such factors include (alone or in combination) Interleukins 1, 2, 4, 5, 6, 7, 10, 12 and 15; granulocyte-macrophage colony stimulating factor, granulocyte colony stimulating factor; a fusion protein comprising Interleukin-3 and granulocyte-macrophage colony stimulating factor; Interferon-gamma; TNF, TGF-β, FL, and biologically active derivatives thereof. A particularly preferred cytokine is CD40L. A soluble form of CD40L is described in U.S. Pat. No. 5,962,406, issued Oct. 5, 1999, as well as U.S. Pat. No. 6,087,329, issued Jul. 11, 2000. Other cytokines will also be useful, as described herein. DNA encoding such cytokines will also be useful in the inventive methods, for example, by transfecting the dendritic cells to express the cytokines. Administration of these immunomodulatory molecules includes simultaneous, separate or sequential administration with the cells of the present invention.

Advantageously, the inventive compositions are administered in the form of a composition comprising at least one cytokine or other immunoregulatory molecule and one or more additional components such as a physiologically acceptable carrier, excipient or diluent. The present invention provides such compositions comprising an effective amount of cytokine or other immunoregulatory molecule, for use in the methods provided herein. The compositions contain agent(s) in any of the forms described herein. The agent may be a cytokine or other immunoregulatory molecule, or an engineered derivative thereof, for example. Several suitable cytokines and immunoregulatory molecules are described herein; others are known in the art.

Compositions may, for example, comprise an agent together with a buffer, antioxidant such as ascorbic acid, low molecular weight polypeptide (such as those having fewer than 10 amino acids), protein, amino acid, carbohydrate such as glucose, sucrose or dextrins, chelating agents such as EDTA, glutathione, and other stabilizers and excipients. Neutral buffered saline or saline mixed with conspecific serum albumin are examples of appropriate diluents. In accordance with appropriate industry standards, preservatives such as benzyl alcohol may also be added. The composition may be formulated as a lyophilizate using appropriate excipient solutions (e.g., sucrose) as diluents. Suitable components are nontoxic to recipients at the dosages and concentrations employed. Further examples of components that may be employed in pharmaceutical formulations are presented in Remington's Pharmaceutical Sciences, 16^{th} Ed., Mack Publishing Company, Easton, Pa., 1980.

Kits for use by medical practitioners include suitable cytokines or other immunoregulatory molecules and a label or other instructions for use in treating any of the conditions discussed herein. The kit preferably includes a sterile preparation of one or more agents (i.e., cytokines or other immunoregulatory molecules), which may be in the form of a composition as disclosed above, and may be in one or more vials.

Dosages and the frequency of administration may vary according to such factors as the route of administration, the particular agent employed, the nature and severity of the disease to be treated, whether the condition is acute or chronic, and the size and general condition of the patient. Appropriate dosages can be determined by procedures known in the pertinent art, e.g. in clinical trials that may involve dose escalation studies.

The inventive compositions may be administered once, or repeatedly. In particular embodiments, a composition is administered over a period of at least a month or more, e.g., for one, two, or three months or even indefinitely. For treating chronic conditions, long-term treatment is generally most effective. However, for treating acute conditions, administration for shorter periods, e.g. from one to six weeks, may be sufficient. In general, the composition is administered until the patient manifests a medically relevant degree of improvement over baseline for the chosen indicator or indicators.

Particular embodiments of the present invention involve administering the inventive composition, comprising a cytokine or other immunoregulatory molecule at a dosage of from about 1 ng/kg/day to about 10 mg/kg/day, more preferably from about 500 ng/kg/day to about 5 mg/kg/day, and most preferably from about 5 micrograms/kg/day to about 2 mg/kg/day, to a patient. In additional embodiments, the cytokine or other immunoregulatory molecule is administered to adults one time per week, two times per week, or three or more times per week, to treat the medical disorders disclosed herein. If injected, the effective amount of cytokine or other immunoregulatory molecule per adult dose may range from 1-20 mg/m², and preferably is about 5-12 mg/m². Alternatively, a flat dose may be administered; the amount may range from 5-100 mg/dose. One range for a flat dose is about 20-30 mg per dose. In one embodiment of the invention, a flat dose of 25 mg/dose is repeatedly administered by injection. If a route of administration other than injection is used, the dose is appropriately adjusted in accordance with standard medical practices. One example of a therapeutic regimen involves injecting a dose of about 20-30 mg of cytokine or other immunoregulatory molecule one to three times per week over a period of at least three weeks, though treatment for longer periods may be necessary to induce the desired degree of improvement. For pediatric patients (age 4-17), one suitable regimen involves the subcutaneous injection of 0.4 mg/kg, up to a maximum dose of 25 mg of cytokine or other immunoregulatory molecule, administered two or three times per week.

The inventive composition(s) is(are) administered to the patient in an amount and for a time sufficient to induce an improvement, preferably a sustained improvement, in at least one indicator that reflects the severity of the disorder that is being treated. Various indicators that reflect the extent of the patient's illness may be assessed for determining whether the amount and time of the treatment is sufficient. Such indicators include, for example, clinically recognized indicators of disease severity, symptoms, or manifestations of the disorder in question. In most instances, an improvement is considered to be sustained if the patient exhibits the improvement on at least two occasions separated by two to four weeks. The degree of improvement generally is determined by the patient's physician, who may make this determination based on signs or symptoms, and who may also employ questionnaires that are administered to the patient, such as quality-of-life questionnaires developed for a given disease.

The relevant disclosures of all publications cited herein are specifically incorporated by reference. The following examples are provided to illustrate particular embodiments and not to limit the scope of the invention.

### EXAMPLE 1

This Example describes a method for generating purified dendritic cells ex vivo. Human bone marrow is obtained, and cells having a CD34⁺ phenotype are isolated and cells are cultured in a suitable medium, for example, McCoy's enhanced media, that contains cytokines that promote the growth of dendritic cells (i.e., 20 ng/ml each of GM-CSF, IL-4, TNF-alpha, or 100 ng/ml FL or c-kit ligand, or combinations thereof). The culture is continued for approximately two weeks at 37° C. in 10% CO₂ in humid air. Cells then are sorted by flow cytometry using antibodies for CD1a⁺, HLA-DR⁺ and CD86⁺. A combination of GM-CSF, IL-4 and TNF-alpha can yield a six to seven-fold increase in the number of cells obtained after two weeks of culture, of which 50-80% of cells are CD1a⁺ HLA-DR⁺ CD86⁺. The addition of FL and/or c-kit ligand further enhances the expansion of total cells, and therefore of the dendritic cells. Phenotypic analysis of cells isolated and cultured under these conditions indicates that between 60-70% of the cells are HLA-DR⁺, CD86⁺, with 40-50% of the cells expressing CD1a in all factor combinations examined.

### EXAMPLE 2

This Example describes a method for collecting and expanding dendritic cells. Prior to cell collection, FL or sargramostim (Leukine®, Immunex Corporation, Seattle, Wash.) may be administered to an individual to mobilize or increase the numbers of circulating PBPC and PBSC. Other growth factors such as CSF-1, GM-CSF, c-kit ligand, G-CSF, EPO, IL-1, IL-2, IL-3, IL-4, IL-5, IL-6, IL-7, IL-8, IL-9, IL-10, IL-11, IL-12, IL-13, IL-14, IL-15, GM-CSF/IL-3 fusion proteins, LIF, FGF and combinations thereof, can be likewise administered in sequence, or in concurrent combination with FL.

Mobilized or non-mobilized PBPC and PBSC are collected using apheresis procedures known in the art. See, for example, Bishop et al., Blood, vol. 83, No. 2, pp. 610-616 (1994). Briefly, PBPC and PBSC are collected using conventional devices, for example, a Haemonetics Model V50 apheresis device (Haemonetics, Braintree, Mass.). Four-hour collections are performed typically no more than five times weekly until approximately 6.5×10⁸ mononuclear cells (MNC)/kg individual are collected.

Aliquots of collected PBPC and PBSC are assayed for granulocyte-macrophage colony-forming unit (CFU-GM) content. Briefly, MNC (approximately 300,000) are isolated, cultured at 37° C. in 5% CO₂ in fully humidified air for about two weeks in modified McCoy's 5A medium, 0.3% agar, 200 U/ml recombinant human GM-CSF, 200 u/ml recombinant human IL-3, and 200 u/ml recombinant human G-CSF. Other cytokines, including FL or GM-CSF/IL-3 fusion molecules (PIXY 321), may be added to the cultures. These cultures are stained with Wright's stain, and CFU-GM colonies are scored using a dissecting microscope (Ward et al., Exp. Hematol., 16:358 (1988). Alternatively, CFU-GM colonies can be assayed using the CD34/CD33 flow cytometry method of Siena et al., Blood, Vol. 77, No. 2, pp 400-409 (1991), or any other method known in the art.

CFU-GM containing cultures are frozen in a controlled rate freezer (e.g., Cryo-Med, Mt. Clemens, Mich.), then stored in the vapor phase of liquid nitrogen. Ten percent dimethylsulfoxide can be used as a cryoprotectant. After all collections from the individual have been made, CFU-GM containing cultures are thawed and pooled, then contacted with FL either alone, sequentially or in concurrent combination with other cytokines listed above to drive the CFU-GM to dendritic cell lineage. The dendritic cells are cultured and analyzed for percentage of cells displaying selected markers as described above.

### EXAMPLE 3

This example illustrates the ability of dendritic cells to stimulate antigen-specific proliferation of T cells. Cell are obtained from an individual who is reactive against C. neoformans. CD34⁺ cells are isolated, cultured for two weeks in the presence of selected cytokines, and isolated by flow cytometry substantially as described in Example 1. The dendritic cells are pulsed with cryptococcal cell wall/membrane (CCWM) antigen (Mody et al., J. Infectious Disease 178:803; 1998), at 37° C. in a 10% CO₂ atmosphere for from 4 to 24 hours, then cultured for an additional 24 hours in the presence or absence of a soluble trimeric form of CD40L (1 μg/ml), in McCoy's enhanced media containing cytokines that support the growth of dendritic cells.

Autologous CCWM-reactive T cells are derived by culturing CD34⁻ cells from the C. neoformans-reactive individual in the presence of CCWM and low concentrations of IL-2 and IL-7 (2 ng/ml and 5 ng/ml, respectively) for two weeks. The CD34⁻ population contains a percentage of T cells (about 5%), a proportion of which are reactive against CCWM, as well as other cell types that act as antigen presenting cells. By week 2, the population of cells will comprise about 90% T cells, the majority of which will be CCWM-specific, with low levels of the T cell activation markers.

Antigen specific T cell proliferation assays are conducted with the CCWM-specific T cells, in RPMI with added 10% heat-inactivated fetal bovine serum (FBS), in the presence of the CCWM-pulsed dendritic cells, at 37° C. in a 10% CO₂ atmosphere. Approximately 1×10⁵ T cells per well are cultured in triplicate in round-bottomed 96-well microtiter plates (Corning) for five days, in the presence of a titrated number of dendritic cells. The cells are pulsed with 1 microCi/well of tritiated thymidine (25 Ci/nmole, Amersham, Arlington Heights, Ill.) for the final four to eight hours of culture. Cells are harvested onto glass fiber discs with an automated cell harvester and incorporated cpm were measured by liquid scintillation spectrometry.

1. A population of activated, C. neoformans antigen-presenting dendritic cells, produced by the process of
(a) obtaining dendritic cells;
(b) causing the dendritic cells to present the antigen by either (i) exposing the dendritic cells to antigen that requires processing in culture under conditions promoting uptake and processing of the antigen, (ii) exposing dendritic cells to peptide antigen under conditions promoting binding of the peptide antigen, or (iii) transfecting the dendritic cells with a nucleic acid encoding the antigen; and
(c) maturing the dendritic cells by exposing them to a CD40 binding protein, wherein dendritic cells that are exposed to peptide antigen are matured prior to exposure, and dendritic cells that are exposed to antigen that requires processing or that are transfected with nucleic acid encoding antigen are matured after antigen has been processed.

2. The population according to claim 1 wherein dendritic cells are obtained by contacting hematopoietic stem or progenitor cells with a molecule selected from the group consisting of GM-CSF, FL, IL-4, TNF-alpha, IL-3, c-kit ligand, fusions of GM-CSF and IL-3, and combinations thereof.

3. The population according to claim 1, wherein the CD40 binding protein comprises a soluble, oligomeric CD40L selected from the group consisting of:
(a) a peptide comprising amino acids 1 through 261, 35 through 261, 34 through 225, 113 through 261, 113 through 225, 120 through 261, or 120 through 225 of human CD40L;
(b) fragments of a peptide according to (a) that bind CD40; and
(c) peptides encoded by DNA which hybridizes to a DNA that encodes a peptide of (a) or (b), under stringent conditions (hybridization in 6×SSC at 63° C. overnight; washing in 3×SSC at 55° C.), and which bind to CD40, and a zipper domain.

4. The population according to claim 3, wherein the soluble, oligomeric CD40 ligand is selected from the group consisting of:
(a) a polypeptide having an amino acid sequence of human CD40L wherein a cysteine at amino acid 194 is replaced with another amino acid; and
(b) a polypeptide that is a fragment of the mutein of (a) that binds CD40;
wherein the amino acid that is substituted for the cysteine at amino acid 194 is selected from the group consisting of tryptophan, serine, aspartic acid, and lysine.

5. A method of stimulating an immune response specific for a C. neoformans antigen in an individual, comprising administering the activated, antigen-expressing dendritic cells of claim 1 to the individual.

6. The method according to claim 5, wherein FL is administered to the individual prior to obtaining the dendritic cells, to expand the number of progenitor cells in the circulation of the individual.

7. The method according to claim 6, wherein the dendritic cells are obtained by obtaining hematopoietic stem or progenitor cells from the individual, and contacting the hematopoietic stem or progenitor cells with a molecule selected from the group consisting of FL, GM-CSF, IL-4, TNF-alpha, IL-3, c-kit ligand, fusions of GM-CSF and IL-3, and combinations thereof.

8. The method according to claim 5, wherein the antigen-expressing, activated dendritic cells are administered simultaneously, sequentially or separately with a molecule selected from the group consisting of Interleukins 1, 2, 3, 4, 5, 6, 7, 10, 12 and 15; granulocyte-macrophage colony stimulating factor, granulocyte colony stimulating factor; a fusion protein comprising Interleukin-3 and granulocyte-macrophage colony stimulating factor; Interferon-gamma; TNF; TGF-β; FL; CD40 binding protein; biologically active derivatives of these cytokines; and combinations thereof.

9. The method of claim 5, wherein the individual is infected with C. neoformans.

10. A method of stimulating an immune response specific for a C. neoformans antigen in an individual, comprising administering the activated, antigen-expressing dendritic cells of claim 3 to the individual.

11. The method according to claim 10, wherein the dendritic cells are obtained by obtaining hematopoietic stem or progenitor cells from the individual, and contacting the hematopoietic stem or progenitor cells with a molecule selected from the group consisting of FL, GM-CSF, IL-4, TNF-alpha, IL-3, c-kit ligand, fusions of GM-CSF and IL-3, and combinations thereof.

12. The method of claim 10, wherein the individual is infected with C. neoformans.

13. A method of preparing C. neoformans antigen-specific T cells from an individual comprising the steps of:
(a) preparing antigen-presenting dendritic cells according to claim 1; and
(b) allowing the dendritic cells to present the antigen to T cells.

14. The method according to claim 13, wherein the antigen-specific T cells are obtained from the individual, exposed to the antigen-presenting dendritic cells ex vivo, and re-administered to the individual.

15. The method according to claim 13, wherein the CD40 binding protein comprises a soluble, oligomeric CD40 ligand selected from the group consisting of:
(a) a peptide comprising amino acids 1 through 261, 35 through 261, 34 through 225, 113 through 261, 113 through 225, 120 through 261, or 120 through 225 of human CD40L;
(b) fragments of a peptide according to (a) that bind CD40;
(c) peptides encoded by DNA which hybridizes to a DNA that encodes a peptide of (a) or (b), under stringent conditions (hybridization in 6×SSC at 63° C. overnight; washing in 3×SSC at 55° C.), and which bind to CD40;
(d) a polypeptide according to (a) wherein a cysteine at amino acid 194 is replaced with another amino acid selected from the group consisting of tryptophan, serine, aspartic acid, and lysine; and
(e) a fragment of the polypeptide of (d) which binds CD40; and a zipper domain.

16. A population of antigen-specific T cells produced by the process of claim 13.

17. The population according to claim 16, wherein the CD40 binding protein comprises a soluble, oligomeric CD40 ligand selected from the group consisting of:
(a) a peptide comprising amino acids 1 through 261, 35 through 261, 34 through 225, 113 through 261, 113 through 225, 120 through 261, or 120 through 225 of human CD40L;
(b) fragments of a peptide according to (a) that bind CD40;
(c) peptides encoded by DNA which hybridizes to a DNA that encodes a peptide of (a) or (b), under stringent conditions (hybridization in 6×SSC at 63° C. overnight; washing in 3×SSC at 55° C.), and which bind to CD40;
(d) a polypeptide according to (a) wherein a cysteine at amino acid 194 is replaced with another amino acid selected from the group consisting of tryptophan, serine, aspartic acid, and lysine; and
(e) a fragment of the polypeptide of (d) which binds CD40; and a zipper domain.

18. A kit for, comprising a cytokine or other immunoregulatory molecule and instructions for the use of the cytokine or other immunoregulatory molecule in the preparation of the activated, C. neoformans antigen-presenting dendritic cells.

19. The kit of claim 18, wherein the cytokine or other immunoregulatory molecule is selected from the group consisting of FL, GM-CSF, IL-4, TNF-alpha, IL-3, c-kit ligand, fusions of GM-CSF and IL-3, and combinations thereof.

20. The kit of claim 19, further comprising a cytokine or other regulatory molecule selected from the group consisting of Interleukins 1, 2, 3, 4, 5, 6, 7, 10, 12 and 15; granulocyte-macrophage colony stimulating factor, granulocyte colony stimulating factor; a fusion protein comprising Interleukin-3 and granulocyte-macrophage colony stimulating factor; Interferon-gamma; TNF; TGF-β; FL; CD40 binding protein; biologically active derivatives of these cytokines; and combinations thereof.

21. The kit of claim 20, wherein the CD40 binding protein is a soluble, oligomeric CD40L selected from the group consisting of:
(a) a peptide comprising amino acids 1 through 261, 35 through 261, 34 through 225, 113 through 261, 113 through 225, 120 through 261, or 120 through 225 of human CD40L;
(b) fragments of a peptide according to (a) that bind CD40;
(c) peptides encoded by DNA which hybridizes to a DNA that encodes a peptide of (a) or (b), under stringent conditions (hybridization in 6×SSC at 63° C. overnight; washing in 3×SSC at 55° C.), and which bind to CD40;
(d) a polypeptide having an amino acid sequence of human CD40L wherein a cysteine at amino acid 194 is replaced with another amino acid is selected from the group consisting of tryptophan, serine, aspartic acid, and lysine; and
(e) a polypeptide that is a fragment of the mutein of (d) that binds CD40.


SEQUENCE LISTING (6 sequences)

SEQ ID NO: 1 - protein, 235 aa, Homo sapiens
MTVLAPAWSPTTYLLLLLLLSSGLSGTQDCSFQHSPISSDFAVKIRELSDYLLQDYPVTVASNLQDEELCGGLWRLVLAQRWMERLKTVAGSKMQGLLERVNTEIHFVTKCAFQPPPSCLRFVQTNISRLLQETSEQLVALKPWITRQNFSRCLELQCQPDSSTLPPPWSPRPLEATAPTAPQPPLLLLLLLPVGLLLLAAAWCLHWQRTRRRTPRPGEQVPPVPSPQDLLLVEH

SEQ ID NO: 2 - protein, 162 aa, Homo sapiens
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 4 - protein, 33 aa, Artificial Sequence
  Zipper Peptide
RMKQIEDKIEEILSKIYHIENEIARIKKLIGER

SEQ ID NO: 5 - protein, 281 aa, Homo sapiens [sequence omitted]

SEQ ID NO: 6 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID